(19) United States
(12) Patent Application Publication
(10) Pub. No.: US 2014/0044779 A1
(43) Pub. Date: Feb. 13, 2014

(54) Liquid Carrier for Oral Delivery of a Pharmacologically Active Agent

(71) Applicants: Anders Carlsson, Stockholm (SE); Bengt Herslöf, Stockholm (SE); Jan Holmbäck, Vaxholm (SE)

(72) Inventors: Anders Carlsson, Stockholm (SE); Bengt Herslöf, Stockholm (SE); Jan Holmbäck, Vaxholm (SE)

(73) Assignee: LIPIDOR AB, Stockholm (SE)

(21) Appl. No.: 14/112,387

(22) PCT Filed: Apr. 20, 2012
(86) PCT No.: PCT/SE2012/000054

§ 371 Date: Oct. 17, 2013

(30) Foreign Application Priority Data
Apr. 18, 2011 (SE) 1100292-0
May 2, 2011 (SE) 1100339-0

(51) Int. Cl. A61K 47/44

(52) U.S. Cl. CPC A61K 47/44
USPC 424/455; 514/772; 424/489; 514/20.5; 424/474

(57) ABSTRACT

A fluid carrier comprises first and second substantially immiscible liquids. The first liquid is an open-chain silicone oil of the formula [(CH₃)₃Si—O]—[(CH₂)₂Si—O]ₙ—[Si(CH₃)₃]. The second liquid is a polar lipid material. The first and second liquids are capable of forming an unstable dispersion. The unstable dispersion can be stabilized by adding a powderous solid insoluble in the liquids. The powderous solid is selected from pharmacologically active agent, pharmaceutical excipient, and their mixtures. The stabilized dispersion is of a creamy or ointment-like or mouldable form, and can be filled into capsules or moulded into tablets so as to be fit for peroral administration.

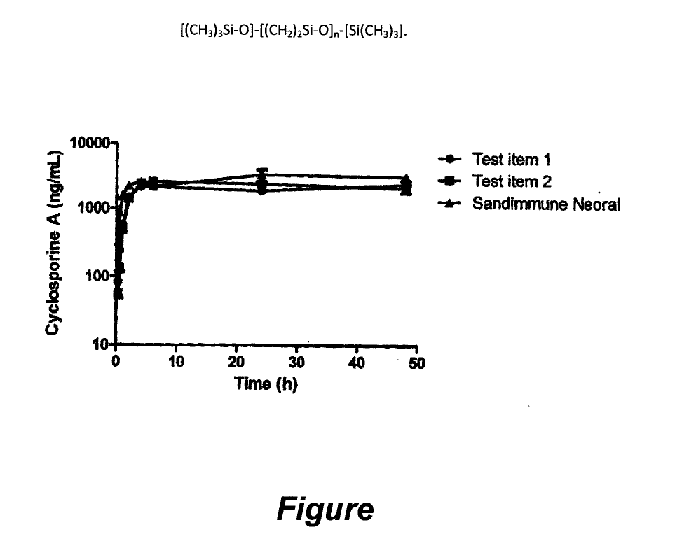

### FIELD OF THE INVENTION

The present invention relates to a liquid carrier for oral delivery of a pharmacologically active agent dissolved or suspended therein. The invention also relates to a method of manufacture of the carrier and a pharmaceutical composition comprising the carrier and a pharmacologically active agent dissolved or suspended therein. Furthermore, the invention relates to uses of the pharmaceutical composition.

### BACKGROUND OF THE INVENTION

Pharmaceutically active agents can be administered perorally dissolved or suspended in a lipid carrier. This kind of administration is advantageous for active agents that are desired not to be released in quantity prior to their passage through the duodenum. It is also useful for active agents, which are poorly soluble or practically insoluble in aqueous liquids but are at least somewhat soluble in lipid carriers.

A problem with lipids is their early and variable (from person to person) degradation during passage through the upper gastrointestinal tract, and the concomitant early and unpredictable release of the active agent. This unpredictability may have contributed to the reluctance to use lipid carriers in the pharmaceutical field.

The degradation of known lipid carriers is due to the sensitivity of their ester linkages to gastric and jejunal lipases. Since there is substantial variation from person to person in regard of the excretion of gastrointestinal enzymes, lipid carriers may be degraded at substantially differing rates by different persons, and their contents thus released in an unpredictable manner.

Moreover, structurally differing triglycerides are degraded in the gastrointestinal tract at substantially different rates (Knutsson L et al., Gastrointestinal metabolism of a vegetable-oil emulsion in healthy subjects. Am J Clin Nutr doi: 10.3945). This suggests that the chemical composition of triglycerides in a lipid carrier should be strictly controlled to improve the gastrointestinal degradation rate of the carrier.

### OBJECTS OF THE INVENTION

One object of the present invention is to provide a liquid carrier of the aforementioned kind, which has improved stability against degradation in the gastrointestinal tract, in particular against degradation in the upper gastrointestinal tract, and a method for its production.

Another object of the present invention is to provide a corresponding pharmaceutical composition and a method for its production.

A further object of the invention is to provide uses of the vehicle and of the composition.

Additional objects of the invention will become evident from the study of the following summary of the invention, the description of preferred embodiments thereof, and the appended claims.

### SUMMARY OF THE INVENTION

According the present invention is disclosed a fluid carrier comprising a first liquid and a second liquid, the first liquid consisting of an open-chain silicone oil of the formula [(CH₃)₃Si—O]—[(CH₂)₂Si—O]ₙ—[Si(CH₃)₃] and the second liquid consisting of or comprising a polar lipid material. The polar lipid material of the second liquid and the non-volatile silicone oil of the first liquid are substantially immiscible. In this application, “substantially immiscible” designates a degree of miscibility of less than 1% by weight, that is, each of the liquids is incapable of dissolving more than 1% by weight of the other liquid. The fluid carrier of the invention is useful in the preparation of pharmaceutical compositions for peroral administration, such as the compositions described below.

When submitting them to a dispersing treatment, such as by a stirring at a high shear rate, the two immiscible liquids of the fluid carrier form unstable dispersions. In this application is understood by “unstable dispersion” a dispersion comprising two immiscible liquids, which separates into its components within a week or a month when stored at room temperature (20° C.).

The non-volatile silicone oil of the first liquid is a dimethicone. Dimethicones are widely used in pharmaceutical and personal care applications. They are mixtures of fully methylated linear siloxane polymers, i.e., polydimethylsiloxanes, and are available in different nominal viscosities, ranging from about 1 cSt (centistoke) to about 100,000 cSt. In the art dimethicones with high viscosities are known to be used in topical formulations as emollient and antifoaming agent. They are also known to be used therapeutically in oral formulations for the treatment of flatulence. Dimethicones with a nominal viscosity of 50 cSt or lower are intended for external use only. The silicone oil of the invention has a viscosity of 50 cSt or more, preferably of 100 cSt or more.

Dimethicones are physiologically and chemically inert materials, which are not metabolized by the body upon oral ingestion. They leave the body unaltered with the faeces. Dimethicones are generally regarded to be essentially non-toxic and non-irritant. They protect the active substance through the upper gastrointestinal tract, whereas the polar lipid material promotes the dissolution of the formulation in the gut as well as the uptake and thereby the oral bioavailability.

The polar lipid material of the second liquid can be described as lipids capable of interaction with water (as defined in D. Small, The Physical Chemistry of Lipids. Plenum Press 1986, section 4.3), for example formed of membrane lipid(s), that is, lipid constituents of biological membranes. Membrane lipids contain a polar, hydrophilic head group and one or more lipophilic hydrocarbon chains. This combination makes the membrane lipid molecules amphipathic and enables them to associate both with water and oil. Such membrane lipids can be classified according to their chemical structure, which is a function of how the polar head group is linked to the lipophilic chains. Sphingolipids (linked by sphingosine) and glycerolipids (linked by glycerol) are the two main groups. Depending on the characteristics of the polar head group sphingolipids and glycerolipids can be further classified as phospholipids comprising a phosphate ester head group and glycolipids comprising a carbohydrate head group. Depending on the specific nature of the carbohydrate group, membrane lipids are sometimes called, for instance, galactolipids, which are glycerolipids with galactose in the polar head group. Examples of common membrane lipids are phosphatidylcholine (PC), phosphatidylethanolamine (PE), and digalactosyldiacylglycerol (DGDG). Membrane lipids of interest can be extracted from, for example, egg yolk (egg lecithin), milk and dairy products, soybeans (soy lecithin), other oil crops, oat kernels, and other cereal and grains. These extracts can be further treated to obtain, for instance, PC from soy beans and galactolipids from oats. Preferred polar lipids are galactolipids, in particular galactolipids from oat kernels, or phospholipids from soybeans (soy lecithin or soy-PC). Examples of synthetic phospholipids comprise dioleoylphosphatidylcholine and dioleoylphosphatidylethanolamine. Other lipids capable of interaction with water are monoglycerides, for example monooleylglycerol.

The first liquid is preferably comprised by the vehicle in an amount of from 50% by weight to 90% by weight. The second liquid is preferably comprised by the vehicle in an amount of from 10% by weight to 50% by weight. It is preferred by the vehicle to not comprise more than 10% of components other than the first and second lipids, more preferred not more than 5% by weight or 2% by weight or even less than 1% by weight.

The fluid carrier of the invention is characterised to comprise two immiscible liquids, which form dispersions that separate into their components in a short time (days to weeks when stored at room temperature).

The fluid carrier of the invention provides for superior incorporation of dry powders, resulting in good stability of the suspensions formed.

According to an important aspect of the invention the fluid carrier, when mixed with a finely dispersed solid, e.g. a fine powder, insoluble in the liquids, forms a stable creamy or ointment—like suspension.

When stored at room temperature, the stable creamy or ointment-like stable suspension of the invention is stable for a month or several months and even for a year or two years or more, that is, does not separate into its components.

Depending on the chemical and physical nature of the solid and its particle size, a minimum amount of the solid is required to form the stable suspension of the invention. For a given substance of given physical and chemical nature as well of particle size, this minimum amount can be easily determined by experimentation, which is within the reach of a person skilled in the art. With some substances, such as hydrocortisone, stable suspensions are obtained by incorporating as little as 3% by weight of the substance into the fluid carrier. A preferred average particle size (with 50% or more of the particles being below average) is one of less than 550 μm or 250 μm, in particular of less than 100 μm or 20 μm, most preferred of less than 5 μm or 2 μm. The stabilizing effect of the particulate solid of the invention can also be obtained by a mixture of particulate substances, such as a particulate pharmacologically or cosmetically active agent, for instance hydrocortisone, and a particulate pharmaceutically or cosmetically acceptable excipient, such as microcrystalline cellulose.

The fluid carrier of the invention comprising a storage-stabilizing amount of a particulate solid incorporated to it is termed first composition of the invention. The incorporated particulate solid can be a pharmacologically active agent or a mixture of pharmacologically active agent and pharmaceutically acceptable excipient. The first composition of the invention is of a creamy or pasty or ointment-like nature. It can be administered orally as such or in a capsule, for instance a hard or soft gelatin capsule.

A pharmaceutically or cosmetically acceptable excipient for use in the invention is preferably a traditional pharmaceutical tablet excipient essentially insoluble in the first and second liquids, that is, of a solubility (w/w) of less than 1.0, 0.5 or 0.1%, preferably of less than 0.01%, selected from filler, binder, glidant, anti-adherent, lubricant, disintegrant, anti-oxidant, and their mixtures. Colorants and flavourings may be used as supplementary excipients in addition to the aforementioned traditional excipients. The excipient can comprise one or more of silicon dioxide, titanium dioxide, aluminium oxide, calcium sulphate, calcium carbonate, dibasic calcium phosphate dihydrate, microcrystalline cellulose, powdered cellulose, cyclodextrin, bentonite, kaolin, lactose, magnesium aluminium silicate, magnesium carbonate, magnesium oxide, magnesium trisilicate, and talc.

According to a further preferred aspect of the invention is disclosed a mouldable second composition of the invention obtained by incorporating an amount of particulate pharmacologically active agent or a combination of pharmacologically active agent and pharmaceutically acceptable excipient into the composition in excess of an amount required for obtaining the stable creamy or ointment-like suspension of the invention. The second composition of the invention is mouldable at room temperature like a dough or potter's clay. The mouldable composition can be extruded from a nozzle, and the extrudate segmented. The segments of the size of a medical tablet for peroral administration can be rounded off mechanically in suitable pharmaceutical equipment after adding an anti-adherent like finely dispersed calcium carbonate, silica or talc. The so obtained tablet cores can be covered with a desired single layer or multi-layered coat, for instance a sugar coat or an enteric coat. The tablets formed are storage-stable, that is, can be stored in a closed container at room temperature for a year or two years or more without suffering a loss of pharmacologically active agent exceeding 5% or 10% by weight. Alternatively, the aforementioned segments or coarse particles of uniform weight of the second mouldable composition can be formed into tablets of uniform shape by pressing them into moulds of desired shape, removing excess composition, and expelling the so formed tablets from the moulds. The mouldable second composition of the invention comprises at least 75% by weight, more preferred at least 85% by weight, and most preferred at least 90% by weight of particulate pharmacologically active agent or a combination of particulate pharmacologically active agent and particulate pharmaceutical excipient.

In the following the invention will be explained in more detail by reference to a drawing and a number of examples.

### DESCRIPTION OF THE FIGURE

The FIGURE illustrates the gastrointestinal absorption of cyclosporine A comprised by a composition of the invention in comparison with two prior art compositions.

### DESCRIPTION OF PREFERRED EMBODIMENTS

Materials.

Dimethicones of different viscosities were obtained from Dow Corning (DC 200 Fluids). Akoline MCM and Capmul MCM EP (medium-chain monoglycerides) were obtained from AAK, Sweden and Abitec Corp., USA, respectively. Tween 80, monoolein (technical grade), cholesterol and hydrocortisone were obtained from Sigma-Aldrich. Potato starch was obtained from KMC (Pharma M20). Dextrose was obtained from Risenta, Sweden. Phosal 50 PG, a standardised mixture of at least 50% by weight of phosphatidylcholine, propylene glycol, sunflower mono- & diglycerides, and ascorbyl palmitate, was obtained from Phospholipid GmbH, Germany. Lipoid S 35 FS and Phospholipon 50 were obtained from Lipoid AG, Switzerland.

TABLE 1
Vehicle composition components

| Short name | Supplier, Trade name | Chemical name, CAS No. | Lot No. |
|---|---|---|---|
| DC 200A | Dow Corning ®, DC 200 | Dimethicone, 9006-65-9 | 5627357 |
| | Fluid 1 000 cSt | | |
| DC 200B | Dow Corning ®, DC 200 | Dimethicone, 9006-65-9 | |
| | Fluid 12 500 cSt | | |
| DC 200C | Dow Corning ®, DC 200 | Dimethicone, 9006-65-9 | |
| | Fluid 100 000 cSt | | |
| Rape seed oil | Eldorado | Food grade rape seed oil purchased | |
| | | in a grocery store | |
| DOPC | Lipoid DOPC | Dioleoylphosphatidylcholine, | 566073-1/32 |
| | | 10015-85-7 | |
| DOPE | Lipoid DOPE | Dioleoylphosphatidylethanolamine, | 656006-01/012 |
| | | 2462-63-7 | |
| MOG | Fluka (Sigma-Aldrich), | Monooleoylglycerol, 25496-72-4 | 1384627 |
| | Monoolein | | |
| MCM | Aarhus Karlshamn, Akoline | Medium chain monoglycerides | 8192270 |
| | MCM | | |
| S100 | Lipoid S100 | Soy bean lecithin, 8002-43-5 | 790551-7/910 |
| S35 | Lipoid S 35 FS | | |
| Phospholipon | Lipoid Phospholipon 50 | | |
| Phosal 50 PG | Phospholipid GmbH | At least 50% phosphatidylcholine, | |
| | | propylene glycol, sunflower mono- & | |
| | | diglycerides, ascorbyl palmitate | |
| Tween 80 | Sigma-Aldrich | Polysorbate 80, 9005-65-6 | |

TABLE 2
Model substances for incorporation into the vehicle of the invention

| Model substance | CAS No | Supplier | Lot No | Wavelength, nm* |
|---|---|---|---|---|
| Cholesterol | 57-88-5 | Sigma-Aldrich | 057K0683 | |
| Hydrocortisone | 50-23-7 | Sigma-Aldrich | 010M1568 | |
| Potato starch | | KMC (Pharma | Sigma | |
| | | M20) | BCBC0008V | |
| Dextrose | 14431-43-7 | Risenta | | |
| Cyanocobalamin (Vitamin B₁₂) | 68-19-9 | Sigma-Aldrich | 030M1567 | 550 |
| Methylene blue | 61-73-4 | Sigma-Aldrich | BCBD4688V | 599 |
| Vanillin | 121-33-5 | Sigma-Aldrich | S80107-209 | 310 |
| Tyrosine | 60-18-4 | Sigma-Aldrich | 1437266V | 278 |

*Wavelength used in the dissolution experiments described below.

### Example 1

### Vehicles

A number of vehicles of the invention are listed in Table 3. Vehicle 6 does not comprise dimethicone and is not a vehicle of the invention.

TABLE 3
Vehicles

| | | | Appearance on standing |
|---|---|---|---|
| No. | Composition | Appearance after mixing | (days to weeks) |
| 1 | 75% DC 200 Fluid 12,500 cSt, | Opaque homogeneous paste, | Two liquid layers |
| | 25% Akoline MCM | ointment-like consistency | |
| 2 | 75% DC 200 Fluid 100,000 cSt, | Opaque homogeneous paste, | Two liquid layers |
| | 25% Akoline MCM | ointment-like consistency | |
| 3 | 75% DC 200 Fluid 12,500 cSt, | Opaque homogeneous paste, | Two liquid layers |
| | 25% Monoolein | ointment-like consistency | |
| 4 | 75% DC 200 Fluid 12,500 cSt, | Opaque homogeneous paste, | Two liquid layers |
| | 25% Tween 80 | ointment-like consistency | |
| 5 | 75% DC 200 Fluid 12,500 cSt, | Opaque homogeneous paste, | Two liquid layers |
| | 25% Tween 80 | ointment-like consistency | |
| 6 | 75% Phosal 50 PG, 25% | Opaque homogeneous paste, | Two liquid layers |
| | Akoline MCM | ointment-like consistency | |
| 7 | 75% DC 200 Fluid 1000 cSt, | Opaque homogeneous paste, | Two liquid layers |
| | 25% Akoline MCM | liquid consistency | |
| 8 | 75% DC 200 Fluid, 500 cSt, 25% | Opaque homogeneous paste, | Two liquid layers |
| | Akoline MCM | liquid consistency | |

Vehicle No. 7 was filled in a hard-gelatin capsule (Licaps, size 1; Capsugel) and stored at room temperature for more than 3 months without any noticeable detrimental effect on the capsule.

### Example 2

### Dispersions of Solid Powderous Agents in Vehicles of Table 3

The powderous agents used are practically insoluble in the vehicles. Mixing of the relatively unstable liquid (pasty) compositions of Table 3 with solid powderous agents resulted in storage-stable creamy suspensions or mouldable masses (Table 4).

TABLE 4
Storage-stable suspensions (compositions of the invention)

| | | | Amount of | |
|---|---|---|---|---|
| | Vehicle | Incorporated | incorporated | |
| No. | (Table 1) | agent | agent | Appearance |
| 1 | 1 | Dextrose | 21% | Smooth paste |
| 2 | 1 | Dextrose | 38% | Smooth paste |
| 3 | 2 | Dextrose | 25% | Smooth viscous paste |
| 4 | 1 | Potato starch | 25% | Smooth paste |
| 5 | 1 | Cholesterol | 25% | Smooth paste |
| 6 | 1 | Hydrocortisone | 3% | Smooth paste |
| 7 | 1 | Cellulose | 93% | Mouldable mass |
| | | powder | 1% | |
| | | Hydrocortisone | | |
| 8 | 5 | Cellulose | 90% | Mouldable mass |
| | | powder | 5% | |
| | | Tyrosine | | |

### Example 3

### Compositions of the Invention Comprising Astaxanthin

In Table 5 several formulations with AstaREAL, a powder rich in the natural antioxidant astaxanthin, are presented. Compositions No. 1 and 2 show that the solubility of AstaREAL in water and ethanol is poor. Compositions No. 3 and 4, containing two phospholipid materials both resulted in slurries, which sedimented on standing. On the other hand, compositions No. 5 to 10 were all stable for several months and may be administered orally to a mammal, either by mixing with food and/or a foodstuff, or by means of capsules or syringes.

TABLE 5
AstaREAL compositions (batch size 5-10 g if not otherwise stated)

| | | AstaREAL | | | Appearance, | Appearance, |
|---|---|---|---|---|---|---|
| No. | Batch | (% by w.) | Vehicle | Treatment | at start | 4 months at 20° C. |
| 1 | ACA100511-1 | 24.2 | Water | Mixing with | Dark red slurry | Sediment, clear |
| | | | | spatula | | supernatant |
| 2 | ACA100511-2 | 24.7 | EtOH abs. | Mixing with | Dark red slurry | Sediment, dark |
| | | | | spatula | | red supernatant |
| 3 | ACA100514-1 | 18.2 | 75% Lipoid S 35 FS, | Dissolution in | Dark red viscous | Dark red slurry |
| | | | 25% Akoline MCM | EtOH abs.; rotary | slurry | with sediment |
| | | | | evaporation | | |
| 4 | ACA100514-2 | 19.7 | 75% Akoline MCM, | Dissolution in | Dark red viscous | Dark red slurry |
| | | | 25% Phospholipon | ETOH abs.; rotary | slurry | with sediment |
| | | | 50 | evaporation | | |
| 5 | ACA100526-2 | 21.2 | 75% DC 200 Fluid | Mixing with | Dark red slurry, | Unchanged |
| | | | 100,000 cSt, 25% | spatula | smooth ointment- | |
| | | | Akoline MCM | | like consistency | |
| 6 | ACA100527-1 | 17.7 | 50% DC 200 Fluid | Mixing with | Dark red slurry, | Unchanged |
| | | | 100,000 cSt, 50% | spatula | ointment-like | |
| | | | Akoline MCM | | consistency, signs | |
| | | | | | of separ. | |
| 7 | ACA100527-2 | 17.6 | 75% DC 200 Fluid | Mixing with | Dark red slurry, | Unchanged |
| | | | 12,500 cSt, 25% | spatula | smooth ointment- | |
| | | | Akoline MCM | | like consistency | |
| 8 | ACA100527-3 | 21.2 | 75% DC 200 Fluid | Mixing with | Dark red slurry, | Unchanged |
| | | | 12,500 cSt, 25% | spatula | firm ointment-like | |
| | | | Phosal 50 PG | | consistency | |
| 9 | ACA100611 | 24.9 | 75% Mineral oil, 25% | Mixing with | Dark red slurry, | Unchanged |
| | | | Akoline MCM | spatula | smooth | |
| | | | | | consistency | |
| 10 | ACA100922 | 19.5 | 65% DC 200 Fluid | Mixing with | Dark red slurry, | Unchanged |
| | (500 g) | | 12,500 cSt, 5% DC | spatula and | smooth ointment- | |
| | | | 200 Fluid 1 000 cSt, | spoon | like consistency | |
| | | | 5% DC 200 Fluid | | | |
| | | | 100,000 cSt, 25% | | | |
| | | | Akoline MCM | | | |

### Example 4

### Dissolution Testing of Model Substances in Vehicles of the Invention

The dissolution behavior of compositions prepared according to the invention was studied according to the following procedure.

Carriers of the invention were prepared by mixing silicone oil and lipid, and by mixing silicone oil, lipid and ethanol. A weighed amount of the model substance was added to the mixture. If necessary, the model substance was ground in a mortar prior to addition in order to obtain sufficiently small particles. If ethanol had been added when preparing the carrier it was evaporated in a rotary evaporator. The composition of the invention was obtained in form of a paste-like to semi-fluid suspension.

A 250 ml beaker was filled with 200 ml of deionised water and placed on a magnetic stirrer with temperature control. The temperature in the dissolution medium was set to 37° C. and the stirring rate to 114 rpm. The fluid in the beaker was continuously sampled by means of a capillary tube and a peristaltic pump (Gilson Minipulse 3) and passed through a UV detector (Shimadzu SPD-10A), and returned to the beaker. When a stable baseline had been obtained, 200 mg of the formulation was added to the beaker. The absorbance was continuously recorded. The half life (t_{1/2}) of model substance release from the formulation was calculated as the time required for reaching half the expected absorbance of the total amount of added substance.

As can be seen from the large differences in half live observed in the dissolution experiments presented in Tables 6-8, the ratio of lipid to silicone oil can substantially affect dissolution (release) behavior. Furthermore, there is a great variation in dissolution behavior of different model substances incorporated in one and the same carrier. The experiments also demonstrate that the nature of the oily component (silicone or triglyceride oil) substantially effects dissolution (Table 6).

TABLE 6
Release of tyrosine from silicone oil/polar lipid/tyrosine compositions of the invention

| Composition | % DC 200B | Lipid 1 | % Lipid 1 | Lipid 2 | % Lipid 2 | % Tyrosine | t_{1/2} (min) |
|---|---|---|---|---|---|---|---|
| KL-01d-1 | 67.6% | MCM | 23.8% | | | 8.6% | 13 |
| KL-01d-3 | 69.0% | MCM | 12.0% | MOG | 10.3% | 8.7% | 79 |
| KL-01d-4 | 65.5%¹ | MCM | 14.2% | 100 | 11.0% | 9.3% | 459 |
| KL-01n-2 | 67.7%² | MCM | 12.1% | DOPE | 11.2% | 9.0% | 15633 |
| KL-01d-2 | 69.0% | MOG | 21.5% | | | 9.5% | 711 |
| KL-01d-5 | 66.9%³ | MOG | 12.7% | DOPC | 11.9% | 8.5% | 1210 |

¹Ethanol (0.16 g/g silicone oil) was used as mixing aid.
²Ethanol (1.32 g/g silicone oil) was used as mixing aid.
³Ethanol (0.36 g/g silicone oil) was used as mixing aid.

TABLE 7
Release of vanillin from silicone oil/polar lipid/vanillin compositions of the invention

| Composition | % DC 200B | Lipid 1 | % Lipid 1 | Lipid 2 | % Lipid 2 | % Vanillin | t_{1/2} (min) |
|---|---|---|---|---|---|---|---|
| KL-01b-9 | 71.8%¹ | MCM | 13.7% | S100 | 13.3% | 1.2% | 93 |
| KL-01n-3 | 73.5%² | MCM | 12.6% | DOPE | 12.6% | 1.2% | 42 |
| KL-01j-1 | 74.6% | MOG | 24.4% | | | 1.0% | 73 |

¹Ethanol (0.21 g/g silicone oil) was used as mixing aid.
²Ethanol (1.21 g/g silicone oil) was used as mixing aid.

TABLE 8
Release of methylene blue from silicone oil/polar lipid/methylene blue compositions of the invention

| Composition | % DC 200B | Lipid 1 | % Lipid 1 | Lipid 2 | % Lipid 2 | % Methylene blue | t_{1/2} (min) |
|---|---|---|---|---|---|---|---|
| KL-01e-1 | 74.4% | MCM | 24.1% | | | 1.5% | 2 |
| KL-01p-2 | 88.2%¹ | MCM | 5.1% | S100 | 5.5% | 1.1% | 104 |
| KL-01p-1 | 48.8% | MCM | 37.1% | S100 | 12.6% | 1.5% | 6 |
| KL-01e-2 | 74.0% | MOG | 24.6% | | | 1.4% | 99 |
| KL-01e-3 | 75.0% | MCM | 12.2% | MOG | 12.0% | 0.9% | 3 |
| KL-01e-5 | 73.7%² | MOG | 13.4% | DOPC | 11.4% | 1.6% | 1579 |

¹Ethanol (0.16 g/g silicone oil) was used as mixing aid.
²Ethanol (0.49 g/g silicone oil) was used as mixing aid.

TABLE 9
Release of Vitamin 12 (cyanocobalamin) from silicone
oil/polar lipid/cobalamin compositions of the invention

| Composition | Silicone oil | % S. oil | Lipid 1 | % Lipid 1 | Lipid 2 | % Lipid 2 | % Vit. B12 | t_{1/2} (min) |
|---|---|---|---|---|---|---|---|---|
| KL-01g-1 | DC 200B | 73.9% | MCM | 25.6% | | | 0.5% | 2 |
| KL-01g-2 | DC 200B | 75.2% | MOG | 24.2% | | | 0.6% | 270 |
| KL-01m-1 | DC 200B | 74.2%¹ | MOG | 12.6% | S100 | 12.1% | 1.0% | 34 |
| KL-01m-3 | Rape seed oil | 74.2%¹ | MOG | 12.6% | S100 | 12.2% | 1.0% | 1359 |

¹Ethanol (0.20 g/g silicone oil) was used as mixing aid.

### Example 5

### Gastrointestinal Absorption of Cyclosporine A

Gastro-intestinal absorption of cyclosporine comprised by a composition of the invention was compared with the absorption from two prior art compositions.

Commercial Prior Art Composition A.

Sandimmune Neoral, oral solution (Novartis, lot HS5107, expiry date November 2013). The known composition is a clear, low viscous solution of 100 mg cyclosporine A per ml, the excipients consisting of a-tocopherol, water-free ethanol, propylene glycol, corn oil, and macroglycerol hydroxystearate. Prior to use the solution was diluted to 1:1 by weight with 10% (w/w) of aqueous ethanol. Accordingly, the cyclosporine concentration was 50 mg/g. The ethanol content was at least 50 mg/g.

Cyclosporine Stock Solution.

A stock solution of cyclosporine A was prepared by mixing 2.00 g of cyclosporine A USP/EP (Abbot Laboratories) with 2.0 g of 99.9% (w/w) ethanol and ultrasonicating the mixture at 40° C. for a few minutes until a clear oil had been formed. The solution contained about 500 mg/g of cyclosporine A.

Prior Art Composition B.

Sesame oil (45 g) and melted Capmul MCM EP (15 g) were mixed. The clear oily formed (9.00 g) was mixed with 1.00 g cyclosporine stock solution to provide composition B in form of a clear oil.

Composition of the Invention C.

The pharmaceutical carrier was prepared by mixing in a 100 ml glass beaker 45 g of silicone oil, DC 200 Fluid 500 cSt, and 15 g of lipid, Capmul MCM ER Prior to mixing Capmul MCM EP was melted in a microwave oven. Blending 1.00 g of cyclosporine A stock solution and 9.00 g of the mixture rendered a milky emulsion.

Animal Tests.

The concentration of cyclosporine A was determined (LC-MS/MS, Method PHARM 1326) in whole rats after administrating a single oral dose of 100 mg/kg by gavage. Twelve male Sprague Dawley rats of about 200 g weight were divided into three groups of four animals for testing one formulation by group. Blood was sampled at 15 and 30 min, and at 1, 2, 4, 6, 24 and 48 hrs after administration. A control sample was taken prior to administration. Mean cyclosporine A concentrations for each group are illustrated in the FIGURE. Cyclosporine A was absorbed after oral administration from all three formulations.

1. Fluid carrier for preparing pharmaceutical compositions for peroral administration, comprising a first liquid and a second liquid, the first liquid consisting of an open-chain silicone oil of the formula [(CH₃)₃Si—O]—[(CH₂)₂Si—O]ₙ—[Si(CH₃)₃], the second liquid comprising polar lipid material, wherein the first and second liquids are substantially immiscible and capable of forming an unstable dispersion.

2. The fluid carrier of claim 1, wherein the vehicle comprises 50% by weight or more, in particular 80% by weight or more of the first liquid.

3. The fluid carrier of claim 1 in form of an unstable dispersion.

4. The fluid carrier of claim 1, wherein the non-volatile silicone oil is a dimethicone.

5. The fluid carrier of claim 4, wherein the dimethicone has a nominal viscosity of 50 cSt or more, preferably of 100 cSt or more.

6. The fluid carrier from claim 1, wherein the second liquid is comprised by the fluid carrier in an amount of from 10% by weight to 50% by weight.

7. The fluid carrier of claim 1 not comprising more than 10% by weight other than the first and second liquid, more preferred not more than 5% by weight or 2% by weight or even less than 1% by weight.

8. Stable pharmaceutical composition for peroral administration in form of a creamy or ointment-like suspension comprising the fluid carrier of claim 1 and a suspension-stabilizing amount of a powderous solid dispersed therein, wherein the powderous solid is substantially insoluble in the first and second liquids.

9. The composition of claim 8, wherein less than 1.0% by weight or 0.5% by weight or 0.1% by weight of the powderous solid incorporated in the dispersion is dissolved in the first and second liquid.

10. The composition of claim 8, wherein the powderous solid has an average particle size of less than 550 μm or of less than 250 μm, in particular of less than 100 μm, most preferred of less than 20 μm.

11. The composition of claim 8, wherein the powderous solid is a pharmacologically active agent or a combination of pharmacologically active agent and pharmaceutically acceptable excipient.

12. The composition of claim 8, wherein the pharmacologically active agent is cyclosporine A.

13. Gelatin capsule filled with the composition of claim 8.

14. Stable mouldable pharmaceutical composition for peroral administration comprising the fluid carrier of claim 1 and a suspension-stabilizing amount of a powderous solid dispersed therein, wherein the powderous solid is substantially insoluble in the first and second liquids, comprising a further amount of powderous solid in addition to said suspension-stabilizing amount.

15. The composition of claim 14, wherein less than 1.0% by weight or 0.5% by weight or 0.1% by weight of the powderous solid incorporated in the dispersion is dissolved in the first and second liquids.

16. Tablet or tablet core formed from an aliquot of the composition of claim 14.

17. The tablet core of claim 16 provided with a pharmaceutically acceptable coat.

18. The fluid carrier of claim 1, wherein the second liquid consists of the polar lipid material.

19. The fluid carrier of claim 1, consisting of the first liquid and the second liquid.

20. The fluid carrier of claim 19, wherein the second liquid consists of the polar lipid material.
